(19) United States
(12) Patent Application Publication
(10) Pub. No.: US 2022/0270777 A1
(43) Pub. Date: Aug. 25, 2022

(54) Dynamic Pinhole Aperture for Charged Particle Therapy Systems

(71) Applicant: Mayo Foundation for Medical Education and Research, Rochester, MN (US)

(72) Inventors: Jason M. Holmes, Scottdale, AZ (US); Wei Liu, Scottsdale, AZ (US)

(21) Appl. No.: 17/668,189

(22) Filed: Feb. 9, 2022

Related U.S. Application Data
(60) Provisional application No. 63/147,401, filed on Feb. 9, 2021.

(51) Int. Cl. G21K 1/02; A61N 5/10; G21K 5/04

(52) U.S. Cl. CPC G21K 1/02; A61N 5/1077; G21K 5/04; A61N 2005/1087

(57) ABSTRACT

A dynamic pinhole aperture is configured for use with charged particle therapy systems, such as proton therapy systems. In general, the dynamic pinhole aperture includes a small and mobile pinhole aperture. The dynamic pinhole aperture is designed to be movable with the beam during irradiation, which allows for reducing the size of each discrete spot and, therefore, the target dose penumbra. The dynamic pinhole aperture is carefully designed to balance the reduction of spot sizes (thus target dose penumbra) and the reduction of beam transmission ratios, which allows for the device to be used clinically to treat large tumors.

### CROSS-REFERENCE TO RELATED APPLICATIONS

This application claims the benefit of U.S. Provisional Patent Application Ser. No. 63/147,401, filed on Feb. 9, 2021, and entitled “DYNAMIC PINHOLE APERTURE FOR CHARGED PARTICLE THERAPY SYSTEMS,” which is herein incorporated by reference in its entirety.

### BACKGROUND

Spot scanning proton therapy (SSPT) uses magnetic steering of a narrow proton beam, termed a beamlet, to deliver a dose to a spot inside the patient. SSPT allows for significant flexibility in dose delivery, enabling treatment optimization methods that were previously not permissible. As a result, SSPT offers improved high-dose conformity when compared with passive scattering proton therapy (PSPT) and offers better sparing of organs-at-risk (OARs) in the mid-dose to low-dose range when compared to intensity-modulated x-ray-based therapy.

The conformity of the dose distribution is characterized by the dose penumbra, which is highly related to the spot size. The new generation of proton beam machines usually has a small in-air spot size of 2-6 mm (σ) at the isocenter depending on the beam energy. Due to the limitation of the lowest energy available from the accelerator, range shifters—which usually are placed upstream in the beamline—have to be used to treat shallow tumors. Unfortunately, the introduction of range shifters significantly increases the spot sizes, which in turn enlarges the dose penumbra greatly and results in poor protection of nearby OARs. This issue is especially severe in head and neck (HN) cancer treatment, where tumors are usually shallow and where the number and proximity of OARs (e.g., brainstem and optic-nerve structures) are more pronounced.

To improve the target dose conformity of SSPT plans, the spot size has to be carefully controlled and reduced if possible. The spot size increase due to the range shifter can be minimized by placing it as close to the patient as possible, such as by using a bolus helmet, extended range shifter (ERS), or movable nozzle designs (MND). Other solutions, such as proton mini-beams, have also been proposed to reduce the spot sizes in proton therapy. Even with these methods, the spot sizes can still be too large, and therefore clinically acceptable SSPT plans cannot be generated, yet. In such scenarios, the protection of adjacent OARs, such as brainstem or optic nerve structures, has to be compromised, which results in undesired patient outcomes.

In PSPT, patient-specific apertures have been used for decades to make the dose distribution to be conformal to the tumors. These types of apertures, so-called “static apertures,” are patient-specific blocks that have been milled out to match the largest cross-section of the tumor in the beam-eye view. The static aperture only reduces the dose outside the largest cross section, which limits its effectiveness in some cases. Static apertures have also been suggested for use in SSPT; however, patient-specific static apertures are expensive and time consuming to produce, which makes adaptive re-planning somewhat prohibitive.

Recently, a new concept of dynamic collimation in SSPT has been proposed and used in clinics. Dynamic collimation does not require patient-specific hardware and also allows for dose conformity along the entire depth of the tumor, albeit with increased complexity as compared to a static aperture. A well-known example of a dynamic collimator is the multi-leaf-collimator (MLC) used in photon therapy. The use of MLCs in proton therapy has been investigated; however, their use has been limited since they would need to span a large area and are quite mechanically complex. Additionally, to be useful with small tumors, the MLC blades would have to be very thin. A mechanically simple and affordable dynamic aperture is, therefore, needed in SSPT.

### SUMMARY OF THE DISCLOSURE

The present disclosure addresses the aforementioned drawbacks by providing a dynamic pinhole aperture assembly that includes a plate and a linear stage assembly. The plate has a pinhole aperture formed therein, and is composed of a material sufficient to attenuate transmission of charged particles therethrough. The plate is coupled to the linear stage assembly and includes a first lateral stage configured to move the plate along a first lateral motion axis, a second lateral stage configured to move the plate along a second lateral motion axis, and a depth stage configured to move the plate along a depth motion axis.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 shows an example dynamic pinhole aperture assembly for use with a charged particle therapy system, such as a proton therapy system.

FIGS. 2A and 2B illustrate an example charged particle therapy system that can implement the dynamic pinhole aperture assembly described in the present disclosure.

### DETAILED DESCRIPTION

Described here is a dynamic pinhole aperture for use with radiation therapy systems, such as photon therapy systems (e.g., intensity-modulated x-ray based radiation therapy systems) and charged particle therapy systems, which can include proton therapy systems, heavy ion (e.g., carbon) therapy systems, and the like. In general, the dynamic pinhole aperture includes a small and mobile pinhole aperture, which in some configurations may have a range shifter coupled thereto. The dynamic pinhole aperture is a simple and low-cost dynamic collimator that is designed to be movable with the beam during irradiation, which allows for reducing the size of each discrete spot and, therefore, the target dose penumbra. In some instances, the dynamic pinhole aperture can be referred to as a spot-scanning aperture as it enables scanning a spot-scanning of the radiation therapy beam. Thus, better critical organ protection with only a slight increase of beam-on time can be achieved simultaneously. The dynamic pinhole aperture trims individual spots while traditional static and dynamic apertures trim the edges of the overall treatment field.

Unlike other dynamic collimators—such as proton multi-leaf collimators, which have many degrees of freedom—the simplicity of the dynamic pinhole aperture described in the present disclosure allows for its inclusion in the dose optimization. This is an advantage over other apertures, in which the shape is based on the cross-sections of the tumor only. Additionally, the simplicity of the dynamic pinhole aperture described in the present disclosure allows for easier incorporation into existing ion therapy systems, and is expected to greatly reduce costs as compared with other dynamic apertures. Further, the small dimension of the dynamic pinhole aperture and its freedom in the z-direction allows for it to be as close to the patient as possible. In other currently available dynamic aperture designs, the device is much larger and the position is fixed, which forces the distance of the aperture to the surface of the patient to be suboptimal in many cases, thereby reducing their usefulness.

The dynamic pinhole aperture described in the present disclosure is configured to reduce the spot size of charged particle treatment. For small radii apertures, the dose may be higher at the entrance as compared with larger radii apertures. The overall effect of the dynamic pinhole aperture assembly on the profiles is to reduce the lateral penumbra.

When placed close to the treatment site, there is a measurable advantage for the dynamic pinhole aperture in terms of spot size reduction. The dynamic pinhole aperture described in the present disclosure reduces spot sizes significantly at shallow depths when compared to an optimal range shifter. The optimal range shifter can be defined as a range shifter with a thickness that minimizes the spot size the most at the same distance to the treatment site. The dynamic pinhole aperture can reduce the spot sizes below that of optimal range shifters, even as the dynamic pinhole aperture is moved to 50 mm or so away from the treatment site.

Advantageously, the spot size reduction attainable using the dynamic pinhole apertures described in the present disclosure instead of an optimal thickness range shifter indicate that the dynamic pinhole aperture is a great option for tumors that are less than 60 mm deep. The dynamic pinhole aperture can be particularly advantageous in cases where it can be placed close to the patient, such as less than 100 mm away. For typical devices in the beamline, 100 mm would be too close to the patient. However, the dynamic pinhole aperture can be placed very close to the patient if needed. The dynamic pinhole aperture can also be made relatively small since the beam delivery and the dynamic pinhole aperture placement are synchronized. Additionally, by placing the range shifter directly against the aperture within the dynamic pinhole aperture device, the device can be made compact as compared to a similar device that has a large separation between the range shifter and aperture. The design with the range shifter abutting aperture also makes the increase of the treatment delivery time due to aperture clinically acceptable for large tumors.

Because the dynamic pinhole aperture can be controlled using only three parameters of the aperture position (x, y, z), these parameters can be incorporated into the treatment plan optimization, optimizing the pinhole aperture position to achieve the best sparing of nearby critical organs. This is a unique advantage of the dynamic pinhole apertures described in the present disclosure, as typical dynamic apertures are far too complex to be included prospectively in the optimization calculation. Usually, conventional dynamic apertures are considered retrospectively after the plan is optimized to get a better dose penumbra.

Another design consideration for the dynamic pinhole aperture assembly described in the present disclosure is the beam transmission ratio, which quantifies the fraction of the protons, or other charged particles, that pass through the pinhole aperture. If the beam transmission ratio is too low, then the treatment time might become prohibitive. On the other hand, if the beam transmission ratio is too high, the spot size may not be sufficiently reduced. To measure the beam transmission ratio, the total dose for a given dynamic pinhole aperture assembly configuration at the Bragg peak position can be compared with the total dose with no pinhole aperture, given the same proton or other charged particle fluence.

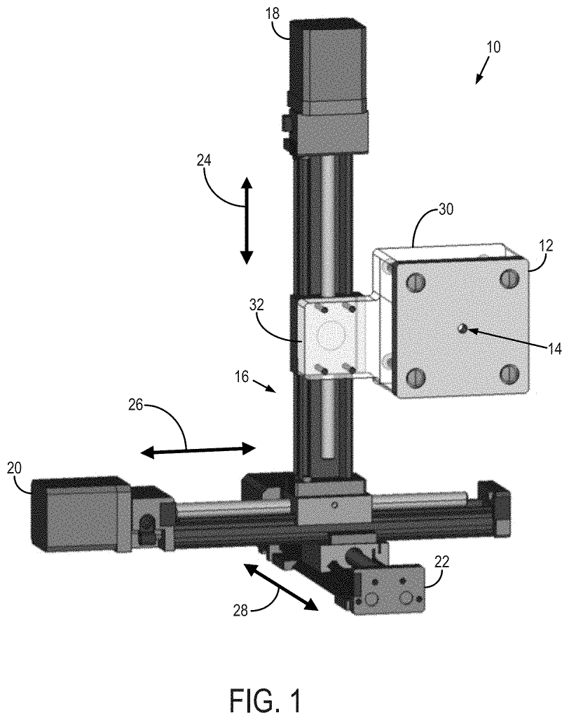

Referring now to FIG. 1, an example dynamic pinhole aperture assembly 10 includes a plate 12 in which a pinhole aperture 14 is formed. The plate 12 is coupled to a linear stage assembly 16, which is configured to translate and accurately position the plate 12 along each of three directions: two horizontal, or lateral, directions (e.g., x and y) and one direction along the beam axis (e.g., the z-direction). The linear stage assembly 16 can, thus, include three linear stages: a first lateral stage 18, a second lateral stage 20, and a depth stage 22. The position of the plate 12 can therefore be translated in each of the x-, y-, and z-directions by translating the plate 12 along a first motion axis 24 corresponding to the motion axis of the first lateral stage 18, a second motion axis 26 corresponding to the motion axis of the second lateral stage 20, and a third motion axis 28 corresponding to the motion axis of the depth stage 22.

The plate 12 can be coupled to a mounting assembly 30, which is then coupled to one of the linear stages of the linear stage assembly 16, such as the first lateral stage 18. The mounting assembly 30 can include a mount 32 to which the plate 12 is coupled, and which is otherwise movably coupled to the linear stage assembly 16 (e.g., by being movably coupled to one of the first lateral stage 18, second lateral stage 20, or depth stage 22). As one example, the plate 12 can be removably coupled to the mounting assembly 30, such that the plate 12 can be used for spots abutting critical organs and moved out of place once finished. Therefore, the increased treatment delivery time due to the plate 12 can be minimized as much as possible, which is advantageous for clinical use to treat large tumors.

The dynamic pinhole aperture assembly 10 can improve dose conformance through the control of the position of the pinhole aperture 14 as opposed to its shape. The dynamic pinhole aperture assembly 10 is designed to scan the pinhole aperture 14 with the charged particle beam, reducing the size of each discrete spot and therefore the target dose penumbra.

As one example, the plate 12 of the dynamic pinhole aperture assembly 10 can be composed of a material that provides suitable attenuation of protons or other charged particles in the energy range to be used by the charged particle therapy system. The plate 12 may be composed, for instance, of metals, metal-containing alloys, or the like. As non-limiting examples, the plate 12 may be composed of materials such as tungsten, nickel, iron, lead, nickel, or brass. If less dense materials, such as brass or lead, are used (e.g., instead of tungsten), it is contemplated that the plate 12 would need to be thicker and the maximum polar angle reduced. The maximum polar angle is related to the off-axis beam transmission ratio, which is relevant for the device to be used to treat large tumors. Tungsten allows for an advantageous balance between a thin plate 12 and large maximum polar angle. A large maximum polar angle allows for a larger off-axis beam transmission ratio and, thus, allows the device to be used over a large tumor while still greatly reducing the discrete spot sizes.

In some configurations, the plate 12 can be dimensioned to reduce the spot sizes for shallow tumors, such as those ranging from 0-10 cm depths. For example, the plate 12 can have a cuboid shape that is 88.9 mm×88.9 mm for its front face, but a smaller or otherwise more optimally shaped plate 12 can also be designed. The thickness of the plate 12 can be designed such that it is thick enough to allow for depths in water of 10 cm, while being thin enough to allow for the pinhole aperture 14 to be relatively small. By having a thinner plate 12, the pinhole aperture 14 can be made relatively small while retaining a large maximum polar angle (thus large off-axis beam transmission ratio), which is advantageous for using the device to treat large tumors. As an example, when the plate 12 is composed of tungsten, the thickness can be on the order of 10 mm. In one non-limiting example, the plate 12 can be composed of tungsten and have a thickness of 11.1 mm. More generally, the plate 12 can have a thickness between 10 mm and 50 mm, depending on the material being used.

Advantageously, the radius of the aperture 14 can be varied based on the type of condition being treated. For example, when treating a patient for cancer, the radius of the aperture 14 can be varied depending on the particular cancer being treated (e.g., eye cancer, brain cancer, etc.). This adjustability of the dynamic pinhole aperture assembly 10 can enable treatment of various cancer types with a single device, which can increase patient throughput and reduce the amount of equipment needed to treat different conditions on the same radiation therapy system.

Taking into account the beam transmission rates for different dynamic pinhole aperture assembly 10 configurations and the corresponding maximum polar angle related to the off-axis beam transmission ratio, a pinhole aperture 14 radius in the range of 2 mm-3 mm can provide an advantageous combination of both high spot size reduction and high beam transmission ratio. For example, a 3 mm radius pinhole aperture 14 may be able to have an on-axis beam transmission ratio of 25-50% to depths less than 100 mm. Therefore, in this example, to deliver the same dose without the dynamic pinhole aperture assembly 10, a proton beam with the same dose rate would have to be on for at least 2-4 times longer. As the dynamic pinhole aperture assembly 10 is used to target spots off the beam-axis, the off-axis beam transmission ratio for a radius of 2 mm and 3 mm can be reduced by less than 50% compared to the value at the middle, even with extreme lateral displacements from the beam axis.

In some configurations, the dynamic pinhole aperture assembly 10 includes a range shifter coupled to the plate 12. For instance, the range shifter can be coupled to the downstream face of the plate 12. In some embodiments, the range shifter can be separated from the face of the plate 12 by an air gap, which in some instances may be a 10 cm air gap. In other embodiments, the range shifter can be in direct contact with the face of the plate 12, or a spacer material can be arranged between the range shifter and the face of the plate 12. When the plate 12 of the dynamic pinhole aperture assembly 10 is close to the patient, the spot size at isocenter is small. As the plate 12 moves farther from the patient (e.g., along the depth direction), the spot size at the isocenter increases. In this way, the spot size can be controlled by the z-position of the plate 12. The x- and y-positions of the spot are similarly controlled by the x- and y-positions of the plate 12 and the pinhole aperture 14. Because the x- and y-positions of the pinhole aperture 14 in the plate 12 are synchronized to the x- and y-position of the spot, the only additional free parameter for the dynamic pinhole aperture assembly 10 is the z-position.

One design consideration for the dynamic pinhole aperture assembly 10 is the effect of the pinhole aperture 14 on the spot size. To characterize the effect of the dynamic pinhole aperture assembly 10 on spot size, proton beams with different energies can be delivered into a water phantom, while varying the pinhole radius and the distance between the distal surface of the pinhole aperture 14 and the surface of the water phantom. The spot size root-mean-square (RMS) can be measured at the Bragg peak position along the beam-axis for different dynamic pinhole aperture assembly 10 configurations.

Another design consideration for the dynamic pinhole aperture assembly 10 is the off-axis beam transmission ratio. To measure the off-axis beam transmission ratio, a larger water phantom can be used, such as a phantom that is 40 cm×40 cm transverse to the beam axis and 10 cm along the beam axis.

As the plate 12 (and thus the pinhole aperture 14) of the dynamic pinhole aperture assembly 10 moves laterally away from the beam axis, the beam polar angle increases, which reduces the beam transmission. At some distance laterally from the beam-axis, the beam can no longer pass through the pinhole aperture 14. The off-axis beam transmission ratio is, therefore, a function of the aspect ratio formed by the pinhole diameter, d, and aperture thickness, t, where the maximum allowable polar angle is θₘₐₓ=tan⁻¹(d/t). To maximize the maximum polar angle, the thickness of the pinhole aperture 14 can be selected to be only as thick as needed to block protons or other charged particles whose energy are high enough to reach a certain depth. For instance, in the non-limiting example described above, the plate 12 can be composed of tungsten and have a thickness on the order of 10-12 mm (e.g., 11 mm, 11.1 mm, 11.11 mm, 11.113 mm, or 11.1125 mm, depending on the desired tolerances) in order to block protons that reach 10 cm deep in water. When the dynamic pinhole aperture assembly 10 is configured for use with shallow tumors, 10 cm WET can be sufficient. For configurations where a deeper tumor is to be treated, a thicker plate 12 can be used.

Taking into account many parameters of the dynamic pinhole aperture assembly 10 described in the present disclosure, the charged particle treatment spot size can be substantially reduced for shallow tumors. Because the dynamic pinhole aperture assembly 10 can be moved for each spot, and because the beam-on time for each spot may be increased, the dynamic pinhole aperture assembly 10 can be advantageously utilized for spots that are adjacent to nearby critical organs. For example, the spots abutting to a nearby critical organ could be delivered with the dynamic pinhole aperture assembly 10 in place and, once finished, the dynamic pinhole aperture assembly 10 can be moved out of view (e.g., by moving the plate 12 out of the beam path), allowing the treatment to resume. As a general device, the dynamic pinhole aperture assembly 10 can be used for any patient with minimal effort. These characteristics make the dynamic pinhole aperture assembly 10 advantageous for reducing the dose penumbra, especially for shallow tumors.

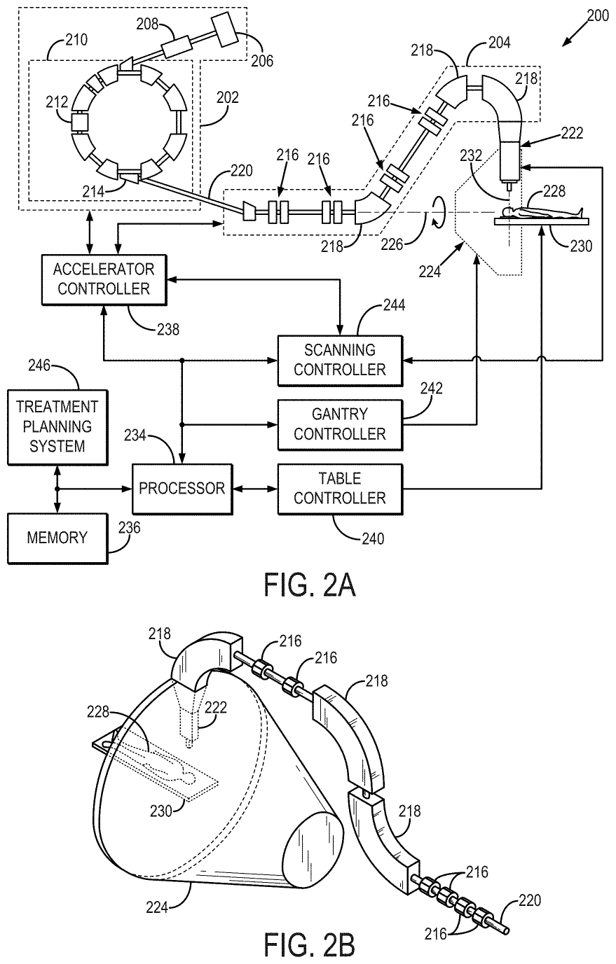

Referring now to FIGS. 2A and 2B, an example of a charged particle therapy system 200, which may be a proton beam therapy system, an ion beam therapy system, or the like, is illustrated. Examples of charged particles for use with a charged particle therapy system include protons, ions, and/or molecules containing such particles. For example, charged particle therapy may include proton therapy and/or ion therapy (e.g., helium ion, carbon ion). An example charged particle therapy system 200 generally includes a charged particle generating system 202 and a beam transport system 204. By way of example, the charged particle generating system 202 may include a synchrotron; however, in other configurations the charged particle generating system 202 may include a cyclotron, a synchrocyclotron, or other suitable accelerator.

The charged particle generating system 202 includes an ion source 206, an injector 208, and an accelerator 210, such as a synchrotron or cyclotron. As a non-limiting example, when the accelerator 210 is a cyclotron, the injector 208 can include an axial injector, a radial injector, or another suitable external injection system suitable for use with a cyclotron. As another non-limiting example, when the accelerator 210 is a synchrotron, the injector 208 can be a linear accelerator (“linac”) or other suitable external injection system.

Ions generated in the ion source 206, such as hydrogen ions (i.e., protons), helium ions, or carbon ions, are accelerated by the injector 208 to form an ion beam that is injected into the accelerator 210. When the accelerator 210 is a synchrotron, the accelerator 210 can provide energy to the injected ion beam by way of an acceleration cavity, where RF energy is applied to the ion beam. In the case of a synchrotron, quadrupole and dipole magnets are used to steer the ion beam about the accelerator 210 a number of times so that the ion beam repeatedly passes through the acceleration cavity.

After the energy of the ion beam traveling in the accelerator 210 has reached a preselected, desired energy level, which would typically be the maximum energy (e.g., 250 MeV for protons), the ion beam is extracted from the accelerator 210 through an extraction deflector 214. Extraction may occur by way of bumping, or kicking, the ion beam to an outer trajectory so that it passes through a septum, or by way of resonance extraction.

The beam transport system 204 includes a plurality of focusing magnets 216 and steering magnets 218. Examples of focusing magnets 216 include quadrupole magnets, and examples of steering magnets 218 include dipole magnets. The focusing magnets 216 and steering magnets 218 are used to contain the ion beam in an evacuated beam transport tube 220 and to deliver the high energy ion beam to a beam delivery device 222 that is situated in a treatment room. In some examples, the beam delivery device 222 may be referred to as a nozzle of the ion therapy system.

The beam delivery device 222 is coupled to a rotatable gantry 224 so that the beam delivery device 222 may be rotated about an axis of rotation 226 to delivery therapeutic radiation to a patient 228 positioned on a patient positioning device 230, which may be a patient table, a patient chair, or the like. The rotatable gantry 224 supports the beam delivery device 222 and deflection optics, including focusing magnets 216 and steering magnets 218, that form a part of the beam transport system 204. These deflection optics rotate about the rotation axis 226 along with the beam delivery device 222. Rotation of the rotatable gantry 224 may be provided, for example, by a motor (not shown in FIGS. 2A and 2B). Alternatively, the beam delivery device 222 can be coupled to one or more fixed beams in one treatment room. In this case, the patient is immobilized on a patient positioning device, such as a robotic chair or table.

In some configurations, the accelerator 210 provides an ion beam to a plurality of beam delivery devices located in different treatment rooms. In such configurations, the beam transport system 204 may connect to a series of switchyards that may include an array of dipole bending magnets that deflect the ion beam to any one of a plurality of deflection optics that each lead to a respective beam delivery device in the respective treatment room.

The beam delivery device 222 is designed to deliver precise dose distributions to a target volume within a patient. In general, an example beam delivery device 222 includes components that may either modify or monitor specific properties of an ion beam in accordance with a treatment plan. For instance, the beam delivery device 222 can include one or more dose monitors (e.g., a main dose monitor and a backup dose monitor). In use, the dose monitor(s) can monitor the dose of the impinging ion beam, and can trigger interlocks that stop beam delivery when deviations from prescribed values are observed. These dose monitors and their associated controls systems can be designed to measure very high beam currents from accelerators, such as cyclotrons, without loss of integrity.

The beam delivery device 222 may also, for example, include a device to spread or otherwise modify the ion beam position and profile, a dispersive element to modify the ion beam energy, and a plurality of beam sensors to monitor such properties. For example, scanning electromagnets may be used to scan the ion beam in orthogonal directions in a plane that is perpendicular to a beam axis 232. Advantageously, as described above the ESS described in the present disclosure are housed within the beam delivery device 222. Because the ESS is capable of selecting the desired energy of the ion beam, the range can be controlled and reduced without the need for a traditional range shifter (“RS”) within the beam delivery device 222. When the beam delivery device 222 is configured for pencil beam-scanning (“PBS”), additional monitors can also be includes in the beam delivery device 222, such as beam profile and spot position monitors. In use, these monitors can trigger interlocks when the ion beam deviates from prescribed values.

The charged particle therapy system 200 is controlled by a central controller that includes a processor 234 and a memory 236 in communication with the processor 234. An accelerator controller 238 is in communication with the processor 234 and is configured to control operational parameters of the charged particle generating system 202, including the accelerator 210 and the beam transport system 204. A table controller 240 is in communication with the processor 234 and is configured to control the position of the patient positioning device (e.g., patient table or chair) 230. A gantry controller 242 is also in communication with the processor 234 and is configured to control the rotation of the rotatable gantry 224. A scanning controller 244 is also in communication with the processor 234 and is configured to control the beam delivery device 222. The memory 236 may store a treatment plan prescribed by a treatment planning system 246 that is in communication with the processor 234 and the memory 236, in addition to control parameters or instructions to be delivered to the accelerator controller 238, the table controller 240, the gantry controller 242, and the scanning controller 244. The memory 236 may also store relevant patient information that may be utilized during a treatment session.

Before the ion beam is provided to the patient 228, the patient 228 is positioned so that the beam axis 232 intersects a treatment volume in accordance with a treatment plan prescribed by a treatment planning system 246. The patient 228 is positioned by way of moving the patient positioning device (e.g. patient table or chair) 230 into the appropriate position. The patient positioning device (e.g., patient table or chair) 230 position is controlled by the table controller 240, which receives instructions from the processor 234 to control the position of the patient positioning device (e.g., patient table or chair) 230. The rotatable gantry 224 is then rotated to a position dictated by the treatment plan so that the ion beam will be provided to the appropriate treatment location in the patient 228. The rotatable gantry 224 is controlled by the gantry controller 242, which receives instructions from the processor 234 to rotate the rotatable gantry 224 to the appropriate position. As indicated above, the position of the ion beam within a plane perpendicular to the beam axis 232 may be changed by the beam delivery device 222. The beam delivery device 222 is instructed to change this scan position of the ion beam by the scanning controller 244, which receives instruction from the processor 234. For example, the scanning controller 244 may control scanning electromagnets located in the beam delivery device 222 to change the scan position of the ion beam.

The dynamic pinhole aperture assembly 10 described in the present disclosure can be incorporated into the beam delivery device 222. In this way, the processor 234 can be configured to control the motion of the plate 12 (and thus pinhole aperture 14) via the scanning controller 244, which is in communication with the processor 234 and is configured to control the beam delivery device 222. For example, the processor 234 can be configured to control the scanning controller 244 to send one or more control signals to a motor control system of the dynamic pinhole aperture assembly 10 to control motion of the linear stages of the linear stage assembly 16. The movement of dynamic pinhole aperture assembly 10 can be facilitated by spot delivery software residing in the memory 236 and implemented by the processor 234, which can provide an estimate of the time-dependent spot delivery sequence in order to move the position of the pinhole more efficiently.

The present disclosure has described one or more preferred embodiments, and it should be appreciated that many equivalents, alternatives, variations, and modifications, aside from those expressly stated, are possible and within the scope of the invention.

1. A dynamic pinhole aperture assembly, comprising:
a plate having a pinhole aperture formed therein, wherein the plate is composed of a material sufficient to attenuate transmission of charged particles therethrough; and
a linear stage assembly coupled to the plate and comprising a first lateral stage configured to move the plate along a first lateral motion axis, a second lateral stage configured to move the plate along a second lateral motion axis, and a depth stage configured to move the plate along a depth motion axis.

2. The dynamic pinhole aperture assembly of claim 1, wherein the plate is composed of tungsten.

3. The dynamic pinhole aperture assembly of claim 2, wherein the plate has a thickness between 10 mm and 50 mm.

4. The dynamic pinhole aperture assembly of claim 3, wherein the plate has a thickness between 11.0 mm and 11.2 mm.

5. The dynamic pinhole aperture assembly of claim 4, wherein the plate has a thickness of 11.1 mm.

6. The dynamic pinhole aperture assembly of claim 1, wherein the plate is composed of one of iron, lead, nickel, or brass.

7. The dynamic pinhole aperture assembly of claim 1, further comprising a mounting assembly coupling the plate to the linear stage assembly, wherein the plate is removably coupled to the mounting assembly.

8. The dynamic pinhole aperture assembly of claim 1, wherein the pinhole aperture has a radius between 2 mm and 3 mm.

9. The dynamic pinhole aperture assembly of claim 1, further comprising a range shifter coupled to the plate.

10. The dynamic pinhole aperture assembly of claim 9, wherein the range shifter is coupled to a downstream face of the plate.

11. The dynamic pinhole aperture assembly of claim 9, wherein the range shifter is coupled to and separated from the plate by an air gap.

12. The dynamic pinhole aperture assembly of claim 11, wherein the air gap separates the range shifter from the plate by 10 cm.

13. The dynamic pinhole aperture assembly of claim 1, further comprising a motion controller configured to control a motion of the plate in both lateral and depth directions by selectively controlling the linear stage assembly.

14. The dynamic pinhole aperture assembly of claim 13, wherein the motion controller is configured to move the plate synchronous with a charged particle beam produced by a charged particle therapy system, thereby reducing a size of each discrete spot of the charged particle beam.
